SEQUENCE LISTING (45 sequences)

SEQ ID NO: 1 - DNA, 20 bp, Artificial
  Synthesized
ccatcctcaaagagctggag

SEQ ID NO: 2 - DNA, 19 bp, Artificial
  Synthesized
tgttgtgctgctgcaggta

SEQ ID NO: 3 - DNA, 18 bp, Artificial
  Synthesized
catcctgcgtctggacct

SEQ ID NO: 4 - DNA, 20 bp, Artificial
  Synthesized
gtacttgcgctcaggaggag

SEQ ID NO: 5 - DNA, 32 bp, Artificial
  Synthesized
ggaagatctcgagccatggtttctaaactgag

SEQ ID NO: 6 - DNA, 30 bp, Artificial
  Synthesized
cggggtaccttactgggaggaagaggccat

SEQ ID NO: 7 - DNA, 19 bp, Artificial
  Synthesized
gggtactccttgttgttgc

SEQ ID NO: 8 - DNA, 21 bp, Artificial
  Synthesized
aaatcccagaactcagagaac

SEQ ID NO: 9 - DNA, 20 bp, Artificial
  Synthesized
ggctccatgactgtgggatc

SEQ ID NO: 10 - DNA, 20 bp, Artificial
  Synthesized
ttcagctgcacagcccagaa

SEQ ID NO: 11 - DNA, 20 bp, Artificial
  Synthesized
agcctaaggcagcttgactt

SEQ ID NO: 12 - DNA, 20 bp, Artificial
  Synthesized
ctcgatgaacttcgggatga

SEQ ID NO: 13 - DNA, 20 bp, Artificial
  Synthesized
cctgcttgtatgctggagtc

SEQ ID NO: 14 - DNA, 20 bp, Artificial
  Synthesized
gaaaagtcgttgatgttgga

SEQ ID NO: 15 - DNA, 20 bp, Artificial
  Synthesized
ctggcctctgccatcttctg

SEQ ID NO: 16 - DNA, 21 bp, Artificial
  Synthesized
ttagcctccttgctcacatgc

SEQ ID NO: 17 - DNA, 22 bp, Artificial
  Synthesized
gtgtccctctagtctatgaagc

SEQ ID NO: 18 - DNA, 22 bp, Artificial
  Synthesized
attgacttgatcctccagatac

SEQ ID NO: 19 - DNA, 22 bp, Artificial
  Synthesized
gcgggactggtatttgtgtctg

SEQ ID NO: 20 - DNA, 21 bp, Artificial
  Synthesized
ttagtgacgacagccgtggtg

SEQ ID NO: 21 - DNA, 20 bp, Artificial
  Synthesized
agcttggtggtggatgaaac

SEQ ID NO: 22 - DNA, 20 bp, Artificial
  Synthesized
ccctcttcagcaaagcagac

SEQ ID NO: 23 - DNA, 18 bp, Artificial
  Synthesized
cgtcatagccgacatcct

SEQ ID NO: 24 - DNA, 18 bp, Artificial
  Synthesized
ctccatttccacgagcag

SEQ ID NO: 25 - DNA, 20 bp, Artificial
  Synthesized
agaaggtcaagagcccagag

SEQ ID NO: 26 - DNA, 20 bp, Artificial
  Synthesized
tccaagttccacgaaatagc

SEQ ID NO: 27 - DNA, 17 bp, Artificial
  Synthesized
gcttgcgacacttcacg

SEQ ID NO: 28 - DNA, 20 bp, Artificial
  Synthesized
tcacttcatacagcacgatc

SEQ ID NO: 29 - DNA, 17 bp, Artificial
  Synthesized
gcttgcgacacttcacg

SEQ ID NO: 30 - DNA, 20 bp, Artificial
  Synthesized
tcacttcatacagcacgatc

SEQ ID NO: 31 - DNA, 17 bp, Artificial
  Synthesized
gcttgcgacacttcacg

SEQ ID NO: 32 - DNA, 20 bp, Artificial
  Synthesized
tcacttcatacagcacgatc

SEQ ID NO: 33 - DNA, 20 bp, Artificial
  Synthesized
ttattgctggagctgccttt

SEQ ID NO: 34 - DNA, 20 bp, Artificial
  Synthesized
ctctggtgggttgaggagag

SEQ ID NO: 35 - DNA, 20 bp, Artificial
  Synthesized
cgggaagtcactggaaacat

SEQ ID NO: 36 - DNA, 20 bp, Artificial
  Synthesized
aaacatggcagtgacaccaa

SEQ ID NO: 37 - DNA, 20 bp, Artificial
  Synthesized
aagccgggatctaccatacc

SEQ ID NO: 38 - DNA, 20 bp, Artificial
  Synthesized
cctgcataagcacatcctga

SEQ ID NO: 39 - DNA, 19 bp, Artificial
  Synthesized
accgaggcactcagaggag

SEQ ID NO: 40 - DNA, 19 bp, Artificial
  Synthesized
gccattagcgcatcacagt

SEQ ID NO: 41 - DNA, 19 bp, Artificial
  Synthesized
gaccttcgttgccctctgt

SEQ ID NO: 42 - DNA, 20 bp, Artificial
  Synthesized
tgaggcggtagtaggacagg

SEQ ID NO: 43 - DNA, 20 bp, Artificial
  Synthesized
gaaatggccaaaatcgacag

SEQ ID NO: 44 - DNA, 20 bp, Artificial
  Synthesized
gagtttgggtaaacccggtc

SEQ ID NO: 45 - DNA, 1896 bp, Homo sapiens
atggtttctaaactgagccagctgcagacggagctcctggcggccctgctcgagtcagggctgagcaaagaggcactgatccaggcactgggtgagccggggccctacctcctggctggagaaggccccctggacaagggggagtcctgcggcggcggtcgaggggagctggctgagctgcccaatgggctgggggagactcggggctccgaggacgagacggacgacgatggggaagacttcacgccacccatcctcaaagagctggagaacctcagccctgaggaggcggcccaccagaaagccgtggtggagacccttctgcaggaggacccgtggcgtgtggcgaagatggtcaagtcctacctgcagcagcacaacatcccacagcgggaggtggtcgataccactggcctcaaccagtcccacctgtcccaacacctcaacaagggcactcccatgaagacgcagaagcgggccgccctgtacacctggtacgtccgcaagcagcgagaggtggcgcagcagttcacccatgcagggcagggagggctgattgaagagcccacaggtgatgagctaccaaccaagaaggggcggaggaaccgtttcaagtggggcccagcatcccagcagatcctgttccaggcctatgagaggcagaagaaccctagcaaggaggagcgagagacgctagtggaggagtgcaatagggcggaatgcatccagagaggggtgtccccatcacaggcacaggggctgggctccaacctcgtcacggaggtgcgtgtctacaactggtttgccaaccggcgcaaagaagaagccttccggcacaagctggccatggacacgtacagcgggccccccccagggccaggcccgggacctgcgctgcccgctcacagctcccctggcctgcctccacctgccctctcccccagtaaggtccacggtgtgcgctatggacagcctgcgaccagtgagactgcagaagtaccctcaagcagcggcggtcccttagtgacagtgtctacacccctccaccaagtgtcccccacgggcctggagcccagccacagcctgctgagtacagaagccaagctggtctcagcagctgggggccccctcccccctgtcagcaccctgacagcactgcacagcttggagcagacatccccaggcctcaaccagcagccccagaacctcatcatggcctcacttcctggggtcatgaccatcgggcctggtgagcctgcctccctgggtcctacgttcaccaacacaggtgcctccaccctggtcatcggcctggcctccacgcaggcacagagtgtgccggtcatcaacagcatgggcagcagcctgaccaccctgcagcccgtccagttctcccagccgctgcacccctcctaccagcagccgctcatgccacctgtgcagagccatgtgacccagagccccttcatggccaccatggctcagctgcagagcccccacgccctctacagccacaagcccgaggtggcccagtacacccacacgggcctgctcccgcagactatgctcatcaccgacaccaccaacctgagcgccctggccagcctcacgcccaccaagcaggtcttcacctcagacactgaggcctccagtgagtccgggcttcacacgccggcatctcaggccaccaccctccacgtccccagccaggaccctgccagcatccagcacctgcagccggcccaccggctcagcgccagccccacagtgtcctccagcagcctggtgctgtaccagagctcagactccagcaatggccagagccacctgctgccatccaaccacagcgtcatcgagaccttcatctccacccagatggcctcttcctcccagtaa

(19) United States
(12) Patent Application Publication
(10) Pub. No.: US 2014/0010786 A1
(43) Pub. Date: Jan. 9, 2014

(54) USE OF HEPATOCYTE NUCLEAR FACTOR 1A IN PREPARATION OF DRUG FOR TREATING MALIGNANT SOLID TUMOR DISEASE

(71) Applicants: Weifen Xie, Shanghai (CN); Xin Zeng, Shanghai (CN); Yong Lin, Shanghai (CN)

(72) Inventors: Weifen Xie, Shanghai (CN); Xin Zeng, Shanghai (CN); Yong Lin, Shanghai (CN)

(73) Assignee: SECOND MILITARY MEDICAL UNIVERSITY OF THE PEOPLE'S LIBERATION ARMY, Shanghai (CN)

(21) Appl. No.: 13/824,691

(22) PCT Filed: Oct. 24, 2011
(86) PCT No.: PCT/CN2011/081163

§ 371 Date: Jul. 22, 2013

(30) Foreign Application Priority Data
Nov. 25, 2010 (CN) 201010559047.3

(51) Int. Cl. C07K 14/47

(52) U.S. Cl. CPC C07K 14/47
USPC 424/93.2; 530/350; 536/23.5; 514/19.3; 514/44 R; 435/320.1

(57) ABSTRACT

A use of a hepatocyte nuclear factor 1α gene and/or protein and a recombinant expression vector containing a hepatocyte nuclear factor 1α in preparation of drugs for treating malignant solid tumor diseases and in preparation of differentiation inducing reagents or composition for inducing differentiation of malignant solid tumor cells. The hepatocyte nuclear factor 1α gene can improve the biological properties of tumor cells, and retard the growth of tumor cells, and up-regulation of expression thereof has therapeutic effects on animal models with malignant solid tumors.

### TECHNICAL FIELD

The present invention relates to a use of a hepatocyte nuclear factor 1α, specifically to a use of a hepatocyte nuclear factor 1α in preparation of drugs for treating malignant solid tumor diseases.

### BACKGROUND ART

The therapy of malignant solid tumor is one of the difficulties in clinical treatment, specifically to the unrespectable malignant solid tumor, which is short of an efficient treatment method. The key protein, molecule and gene which are closely related to the development of tumor cells are selected for the specific targeting regulation, which is one of the core problems in treatment of malignant solid tumor. In recent years, with the research of the human genome project continuously goes deeper, the researchers utilize the genetic technology to regulate even change the expression of important cell gene in order to change the phenotype, differentiation condition and biological function thereof, which would make it possible for tumor cell to appear apoptosis, growth retardation thus leading to the anti-tumor effect.

The hepatocyte nuclear factor 1 (HNF1) belongs to the POU-homeodomain family, it is an important transcription protein for regulating the cell differentiation and maintaining the biological function of the hepatocyte, it is expressed at a high level in the matured hepatocyte, in which, HNF1α is an important subtype of the HNF1. The researches on HNF1α gene knockout mice show that: HNF1α is the necessary transcription factor in the growth and development of liver, and closely related to establishing and maintaining the final normal differentiation and development of the fetal liver. The HNF1α gene knockout mice appear serious liver and renal functions damage and most of them die in few days after birth. The HNF1α combine the cis-acting element in the forms of homodimer or heterodimer with HNF1β, and interact with transcription activating protein to change the chromosome structure nearby the promoter or enhancer, thus realizing the regulation for the differentiation and functional genetic expression at transcriptional level. Although foreign studies have reported that the expression level of HNF1α is closely related to the differentiation of hepatocellular carcinoma, after down-regulation the expression of HNF1α in hepatoma cell lines, the expression of some hepatocyte functional genes of tumor cells is reduced; but the effects whether the HNF1α can improve the biological properties of tumor cells and reduce tumor formation of hepatocellular carcinoma and reverse its poorly differentiation condition are not yet confirmed; the regulation effects on other malignant solid tumor are undefined; furthermore, the way through up-regulation the HNF1α expression is not studied as a method for treating malignant solid tumor.

China Application of application No. 200810034200.3 discloses a use of hepatocyte nuclear factor 4α (HNF4α) for the treatment of human malignant solid tumors through induction-differentiation therapy. The invention relates to the use of hepatocyte nuclear factor 4α for the differentiation of human malignant solid tumor cells through induction therapy, thereby the HNF4α is applied in the method and use for treating malignant solid tumor. The research shows that HNF4α can promote the induction-differentiation of tumor cells through regulating the expression of HNF4α gene of malignant solid tumor cells, it provide a new method for the treatment of solid tumors through induction-differentiation therapy. However, a use of a hepatocyte nuclear factor 1α gene and/or protein in preparation of drugs for treating malignant solid tumor diseases has not been reported at present.

### SUMMARY OF THE INVENTION

The first purpose of this invention is aim at the drawbacks of the prior art and to provide a use of a hepatocyte nuclear factor 1α gene and/or protein in preparation of drugs for treating malignant solid tumor diseases.

The second purpose of this invention is to provide a use of a hepatocyte nuclear factor 1α and/or protein in preparation of differentiation inducing reagents or composition for inducing differentiation of malignant solid tumor cells.

The third purpose of this invention is to provide a use of a recombinant expression vector containing a hepatocyte nuclear factor 1α in preparation of drugs for treating malignant solid tumor diseases.

The fourth purpose of this invention is to provide a use of a recombinant expression vector containing a hepatocyte nuclear factor 1α in preparation of differentiation inducing reagents or composition for inducing differentiation of malignant solid tumor cells.

The fifth purpose of this invention is to provide a method for inducing or promoting differentiation of malignant solid tumor of mammals.

To achieve the above first purpose, this invention takes the following technical solutions: a use of a hepatocyte nuclear factor 1α gene and/or protein in preparation of drugs for treating malignant solid tumor diseases.

To achieve the above second purpose, this invention takes the following technical solutions: a use of a hepatocyte nuclear factor 1α and/or protein in preparation of differentiation inducing reagents or composition for inducing differentiation of malignant solid tumor cells.

Said solid tumor is selected from liver cancer, gastric cancer, intestinal cancer, pancreatic cancer, lung cancer, prostate cancer or gonad tumor.

Said composition is pharmaceutical composition.

Said pharmaceutical composition contains (a) the HNF1α protein, the HNF1α coding sequence or the expression vector containing said coding sequence, and (b) the acceptable carrier or excipient in pharmaceutical field.

Said expression vector includes viral vector and non-viral vector.

Said pharmaceutical composition is applied in preparation of drugs for restraining the formation of solid tumor in vivo. Said hepatocyte nuclear factor 1α is the human hepatocyte nuclear factor 1α.

To achieve the above third purpose, this invention takes the following technical solutions: a use of a recombinant expression vector containing a hepatocyte nuclear factor 1α in preparation of drugs for treating malignant solid tumor diseases.

To achieve the above fourth purpose, this invention takes the following technical solutions: a use of a recombinant expression vector containing a hepatocyte nuclear factor 1α in preparation of differentiation inducing reagents or composition for inducing differentiation of malignant solid tumor cells.

Said solid tumor is selected from liver cancer, gastric cancer, intestinal cancer, pancreatic cancer, lung cancer, prostate cancer or gonad tumor.

Said expression vector includes viral vector and non-viral vector.

To achieve the above fifth purpose, this invention takes the following technical solutions: a method for inducing or promoting differentiation of malignant solid tumor of mammals, said method includes the following steps: the objects of mammals that need treatment are supplied with the hepatocyte nuclear factor 1α protein, the HNF1α coding sequence or expression vector containing said coding sequence.

The advantages of this invention are: this invention provides a new use of the hepatocyte nuclear factor 1α and a new therapy for treating malignant solid tumor. By using the genetic engineering technology to regulate the HNF1α gene expression in solid tumor cells, this invention prove that the HNF1α gene can improve the biological properties of tumor cells, and retard the growth of tumor cells, and up-regulation of expression thereof has therapeutic effects on animal models with malignant solid tumors through injecting the HNF1α adenovirus vector. It is a new exploration in the field of malignant solid tumor treatment.

### BRIEF DESCRIPTION OF THE DRAWINGS

FIG. 1. The expression of HNF1α gene in human hepatoma cell lines was detected by Real-time RT-PCR.

FIG. 2. The expressions of HNF1α gene in human hepatocellular carcinoma (HCC) cancerous tissues and surrounding tissues were detected by Real-time RT-PCR.

FIG. 3. The expressions of HNF1α protein in human hepatocellular carcinoma (HCC) cancerous tissues and surrounding tissues were detected according to immunohistochemistry analysis (I: The expression of HNF1α in HCC surrounding tissues was higher than that of the HCC cancerous tissues; II: The expression of HNF1α in HCC surrounding tissues was not apparently altered compared with that of the HCC cancerous tissues; III: The expression of HNF1α in HCC surrounding tissues was lower than that of the HCC cancerous tissues).

FIG. 4. The immunohistochemistry analysis was used to examine HNF1α gene expression in hepatocellular carcinoma model with DEN-treated mice and the expression was down-regulated gradually.

FIG. 5. The cDNA fragment containing HNF1α was obtained by RT-PCR.

FIG. 6. The shuttle plasmid pAdTrack-CMV-HNF1α was obtained through connection in vitro and identified by enzyme digestion with Bgl II and kpn I.

FIG. 7. The recombinant adenovirus plasmid pAdHNF1α was identified by enzyme digestion with Pac I.

FIG. 8. The recombinant adenovirus plasmid pAdHNF1α was identified by enzyme digestion.

FIG. 9. AdHNF1α infect Hep3B (A and B), Huh7 (C and D), MHCC-H (E and F) respectively; and GFP was expressed in MHCC-L (G and H) cell after three days.

FIG. 10. The expression of HNF1α protein of AdHNF1α-infected hepatoma cell was detected by western blot after three days.

FIG. 11. The quantitative analysis of the expression of HNF1α protein of AdHNF1α-infected hepatoma cell was carried out after three days.

FIG. 12. The localization of the expression of HNF1α protein of AdHNF1α-infected hepatoma cell was detected by immunofluorescence after three days.

FIG. 13. The quantitative analysis of the expression of HNF1α gene and the related functional gene mRNA of hepatocyte was carried out in AdHNF1α-infected hepatoma cell lines.

FIG. 14. The apoptosis variation of AdHNF1α-infected hepatoma cell lines was detected after three days.

FIG. 15. The cell cycle variation of AdHNF1α-infected hepatoma cell lines was detected after three days.

FIG. 16. Real-time RT-PCR and western blotting were carried out to examine the influence on cell cycle related protein of AdHNF1α-infected hepatoma cell lines after three days.

FIG. 17. The influence on proliferation ability of different human hepatoma cell lines introduced with exogenous HNF1α.

FIG. 18. The influence on colony formation of different human hepatoma cell lines introduced with exogenous HNF1α.

FIG. 19. The tumorigenicity experiment was carried out through inoculation in vivo with AdHNF1α-infected hepatoma cell Hep3B.

FIG. 20. The tumorigenicity experiment was carried out through inoculation in vivo with AdHNF1α-infected hepatoma cell Huh7.

FIG. 21. The subcutaneous tumorigenicity model was treated by intratumoral injection of AdHNF1α gene.

FIG. 22. The experimental hepatoma model was obtained through orthotopic injection of HNF1α gene in liver to treat hepatoma cell.

### DETAILED DESCRIPTION OF THE PREFERRED EMBODIMENTS

The invention is further described by combining with figures and embodiments, but the protection scope of this invention is not restricted to the scope of disclosure in the following experiments.

### Example 1

### The Expression of HNF1α Gene in Human Hepatoma Cell Lines was Detected by Real-Time RT-PCR

1. The hepatoma cell lines Huh-7, Hep3B, MHCC-H, MHCC-L, PLC, YY and 7721 were inoculated in 6-well plates with 5×10⁵/well, then cultivated in fresh culture solution containing 10% fetal calf serum. The next day, the RNA was extracted from cells and then determined by spectrophotometer at OD₂₆₀. The RNA concentration was made into working concentration (1 μg/μl and 0.1 μg/μl), and the integrity of RNA was detected by 1% agarose gel electrophoresis.

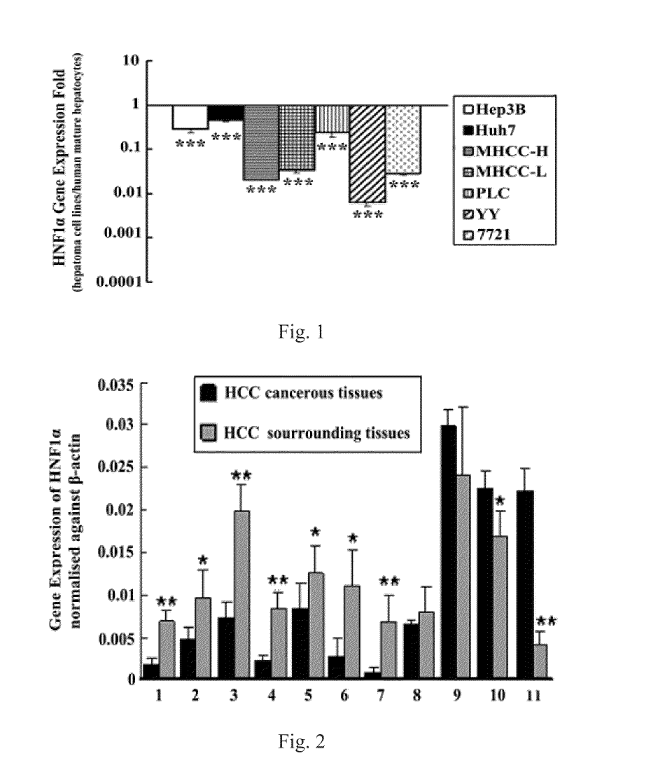

2. Real-time RT-PCR: 4 μg RNA, 2 μl Random primer and DEPC treated water were mixed to 33 μl, after placed at 70° C. for 5 min and 0° C. for 5 min, 10 μl 5× Buffer, 3 μl dNTP, 2 μl RNA reverse transcriptase and 2 μl RNAase inhibitor were added into the mixture. After the aforesaid mixture was mixed and placed at 37° C. for 1.5 h, the reverse transcription product was obtained (see Table 1). The diluted reverse transcription product of 1 μl was taken out as a template for Real-time PCR amplification, the gene primer sequences were shown in Table 2. The reaction condition: pre degenerated at 94° C. for 30 s, and then placed at 94° C. for 10 s and at 60° C. for 30 s, after 40 cycles in all, the solubility curve was detected. The results showed that the expression of HNF1α mRNA of each hepatoma cell line was down-regulated obviously as shown in FIG. 1.

TABLE 1

| Composition | Volume (μl) |
|---|---|
| sense primer | 0.4 μl |
| antisense primer | 0.4 μl |
| reverse transcription | 1 μl |
| product | |
| SYBR | 10 μl |
| ddH₂O | 11.3 μl |

TABLE 2
Human primer sequence

| Gene | Primer sequence |
|---|---|
| HNF1 α | Sense strand 5′-CCATCCTCAAAGAGCTGGAG-3′ (SEQ ID NO. 1) |
| | Antisense strand 5′-TGTTGTGCTGCTGCAGGTA-3′ (SEQ ID NO. 2) |
| β-actin | Sense strand 5′-CATCCTGCGTCTGGACCT-3′ (SEQ ID NO. 3) |
| | Antisense strand 5′-GTACTTGCGCTCAGGAGGAG-3′ (SEQ ID NO. 4) |

### Example 2

### The Expressions of HNF1α Gene and Protein in Human Hepatocellular Carcinoma Cancerous Tissues and Surrounding Tissues were Detected by Real-Time RT-PCR and Immunohistochemistry

1. Real-time RT-PCR: the RNA of human hepatocellular carcinoma cancerous tissues and surrounding tissues was extracted by Trizol method and determined by spectrophotometer at OD₂₆₀. The RNA concentration was made into working concentration (1 μg/μl and 0.1 μg/μl), and the integrity of RNA was detected by 1% agarose gel electrophoresis. 4 μg RNA was carried out reverse transcription and Real-time PCR amplification (the reverse transcription reaction, Real-time PCR reaction condition and primer sequence were same as aforementioned). The results indicated that the expression of HNF1α in hepatocellular carcinoma cancerous tissues was reduced compared with that of surrounding tissues in 7 paired (63.63%) among 11 paired human hepatocellular carcinoma cancerous tissues/surrounding tissues (see FIG. 2).

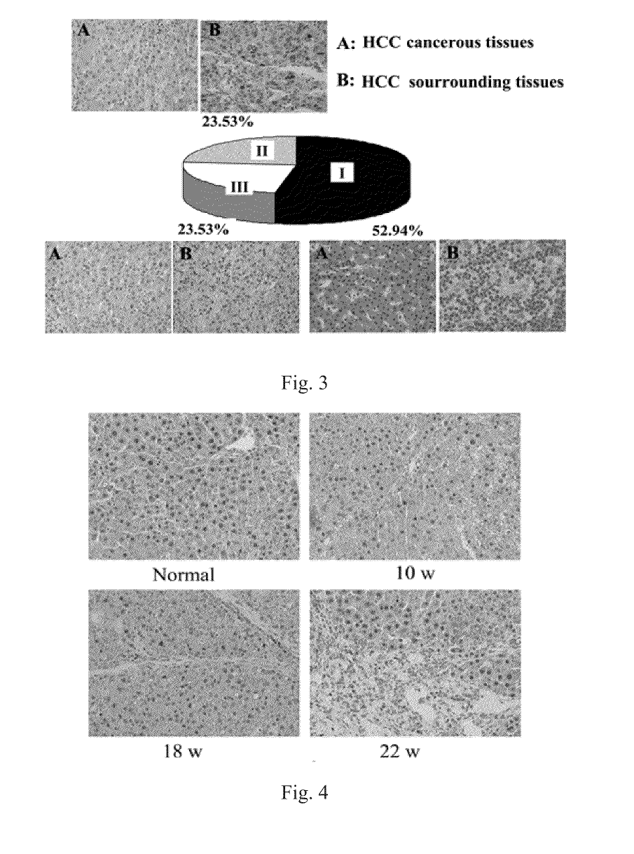

2. Immunohistochemistry: the human hepatocellular carcinoma cancerous tissues and surrounding tissues were performed the wax block 4 mm serial sections, and then fixed in 60° C. oven for 30 min. After dewaxed, the endogenous peroxidase was eliminated from tissues in 3% H₂O₂ at room temperature for 10 min. The microwave antigen repair was conducted in citrate buffer solution, after added 1:10 normal rabbit serum at room temperature for closing 30 min, the HNF1α antibody was added into dropwise and kept at 4° C. for overnight. The next day, the specimen was washed three times using PBS (0.01 M, pH 7.4) for 5 min at a time, then added secondary antibody and incubated at room temperature for 30 min. After the specimen was washed by PBS, SABC (1:100) was added into and incubated at room temperature for 20 min. DAB staining, mounting with common resin and observation under light microscope were performed subsequently. According to the scope of positive staining, the immunohistochemistry sections were analyzed by semi-quantitative using an image analyzer, each section was scanned in four visions. The area of positive staining was measured by image analysis system and the percentage of thereof to total area was calculated automatically. The expression conditions of HNF1α in 17 paired human hepatocellular carcinoma cancerous tissues/surrounding tissues were analyzed. The results displayed that the expression of HNF1α in human hepatocellular carcinoma cancerous tissues was reduced compared with that of surrounding tissues in 52.94% patients (9/17), as shown in FIG. 3.

### Example 3

### The Expressions of HNF1α Gene and Protein in Idiopathic Hepatocellular Carcinoma Model with DEN-Treated Mice were Detected According to Immunohistochemistry Analysis

1. The idiopathic hepatocellular carcinoma model with DEN-treated mice was prepared by intraperitoneal injection of 70 mg/kg DEM. In modeling process, the mice were sacrificed before modeling, 10 w, 18 w and 22 w post-modeling.

2. The hepatic tissue of mice were taken out and fixed in 10% neutral buffered formal in for overnight. The tissue was trimmed into clumps with 1.0×1.0×0.5 cm, soaked under running water for 12 h, dehydrated by gradient ethanol (50%-75%-80%-95%-absolute ethyl alcohol), then made to organization wax block after dimethylbenzene treatment and waxdip. Immunohistochemistry staining was performed after the wax block was sliced continuously. The results exhibited that the expression of HNF1α in mice hepatic tissue was reduced gradually with molding time extension, the expression of HNF1α in hepatocellular carcinoma tissue was the weakest, as shown in FIG. 4.

### Example 4

### The Recombinant Replication-Defective Adenovirus Carrying HNF1α (AdHNF1α) was Constructed

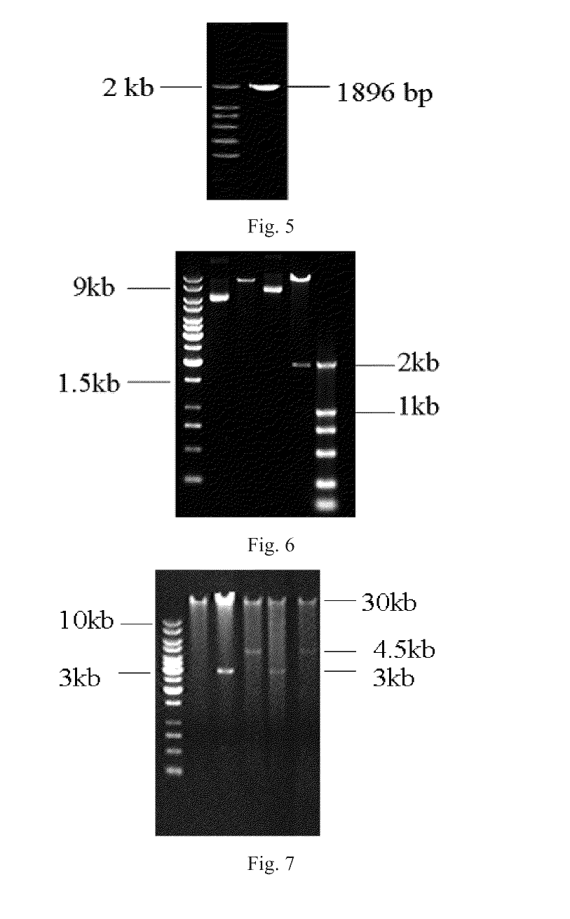

1. The cDNA fragment of HNF1α with 1896 bp was obtained: the primer was designed and synthesized according to cDNA sequence of human HNF1α. The restriction enzyme of Bgl II was inserted into the 5′ end of the sense strand: sense 5′-GGAAGATCTCGAGCCATGGTTTCTAAACTGAG-3′ (SEQ ID NO. 5); the restriction enzyme of Kpn I was inserted into the 5′ end of the antisense strand: antisense 5′-CGGGGTACCTTACTGGGAGGAAGAGGCCAT-3′ (SEQ ID NO. 6). As shown in FIG. 5, the cDNA fragment of HNF1α was obtained by PCR amplification and determined its size by 1% agarose gel electrophoresis. Then the gel was cut, recovered and put into the Eppendorf tube, and weighed it. The NT solution with 200 ml/100 mg gel was added into the Eppendorf tube. The gel was melt at 50° C. for 5-10 min; subsequently the dissolved solution was loaded onto a chromatographic column and then centrifuged at 13000 rpm for 1 min; afterwards, NT3 buffer solution of 600 μl was added into and centrifuged at 13000 rpm for 2 min. The DNA segment was eluted using 30 μl double-distilled water through a chromatographic column, after kept still for 1 min, centrifuged at 13000 rpm for 1 min, the elution was transferred into a clean Eppendorf tube. After spectrophotometer determined the absorbance of previous described elution at OD₂₆₀ₙₘ, the fragment size was identified by 1% agarose gel electrophoresis. It should be noted that the full-length sequence of said cDNA of HNF1α is the said nucleotide sequence in SEQ ID NO. 45.

TABLE 3

| Composition | Volume (μl) |
|---|---|
| sense primer | 2 μl |
| antisense primer | 2 μl |
| normal hepatocyte cDNA | 2 μl |
| PrimerSTAR enzyme | 1 μl |
| 5 × buffer solution | 20 μl |
| dNTP | 8 μl |
| ddH2O | 65 μl |

Reaction condition: at 98° C. for 10 s, 68° C. for 8 min, 35 cycles in all.

2. The adenovirus plasmid pAdHNF1α carrying HNF1α was constructed: the shuttle plasmid pAdTrack-CMV and HNF1α cDNA were digested by Kpn I and Bgl II for 4 h and purified, respectively. The plasmid pAdTrack-CMV of 0.1 μg, HNF1α cDNA of 0.4 μg, 10× T₄ buffer solution of 2 μl, T₄ DNA ligase of 1 μl, (2U) and ddH₂O, total volume of 20 μl, were mixed and connected at 16° C. for overnight. The connected product was added into competent bacteria HD5α for transformation, planking was carried out in the LB medium containing Kanamycin, and then cultivated at 37° C. for overnight. The single bacterial colony was selected for cloning; the bacterial colony containing the amplified cDNA fragment of HNF1α was extracted by Qiagen-tip 100 kit to obtain the plasmid pAdTrack-CMV-HNF1α, and then to identify it, as shown in FIG. 6. The Pme I incision enzyme digested the pAdTrack-CMV-HNF1α in order to make it linearization. The competent BJ5183 bacteria of 20 μl was co-transformed with 0.4 μg linear pAdTrack-CMV-HNF1α and 0.1 μg super helix pAdEasy-1 plasmid by electroporation in the conditions of 2000V, 200 Ohms and 25 μFD. The virus carrying plasmid pAdHNF1α was screened by LB medium containing Kanamycin and identified by sequencing.

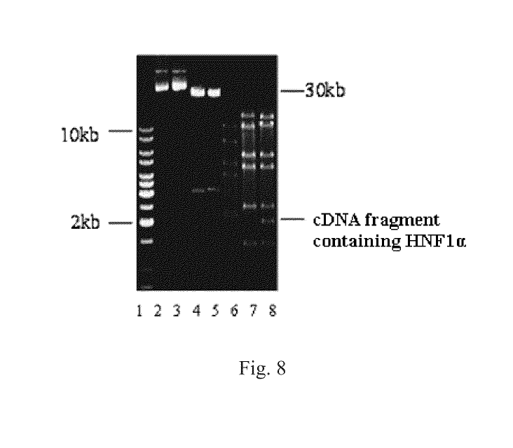

3. The adenovirus AdHNF1α was packed and amplified: the 293 cells were revived and inoculated in 10 cm tissue culture dish with 4.8×10⁶/dish. The cells were cultivated by adding DMEM at 37° C. and 5% CO₂; after 24 h, the cells density reached to 60%˜80%. The pAdHNF1α was digested by Pac I in order to make it linearization and then the serum-free DMEM culture solution of 250 μl was added into the linear pAdHNF1α to prepare the A solution; the Lipofectamin of 20 μl and the serum-free DMEM culture solution of 250 μl were mixed to prepare the B solution. The A solution and B solution were mixed, after kept still at room temperature for 30 min, added into 293 cells for transfection, the culture solution was changed after 4 h. The 293 cells and supernatant were collected after 7 d and repetitively freeze-thawed 4 times in liquid nitrogen and 37° C. water bath. The specimen was centrifuged at 5000 rpm for 5 min; the virus supernatant was collected and the 293 cells were infected again by virus supernatant for amplification; after 2˜3 d, the virus was collected, the steps of infection and collection were repeated, the final collected virus supernatant was subpackaged and determined thereof titer. The AdHNF1α of 1×10¹⁰ efu/ml titer was obtained finally and stored at −80° C., as shown in FIG. 7 and FIG. 8.

### Example 5

### Real-Time RT-PCR, Western Blot and Immunofluorescence were Carried Out to Detect the Expression and Localization of HNF1α in Human Hepatocellular Carcinoma Cell Lines Infected with AdHNF1α

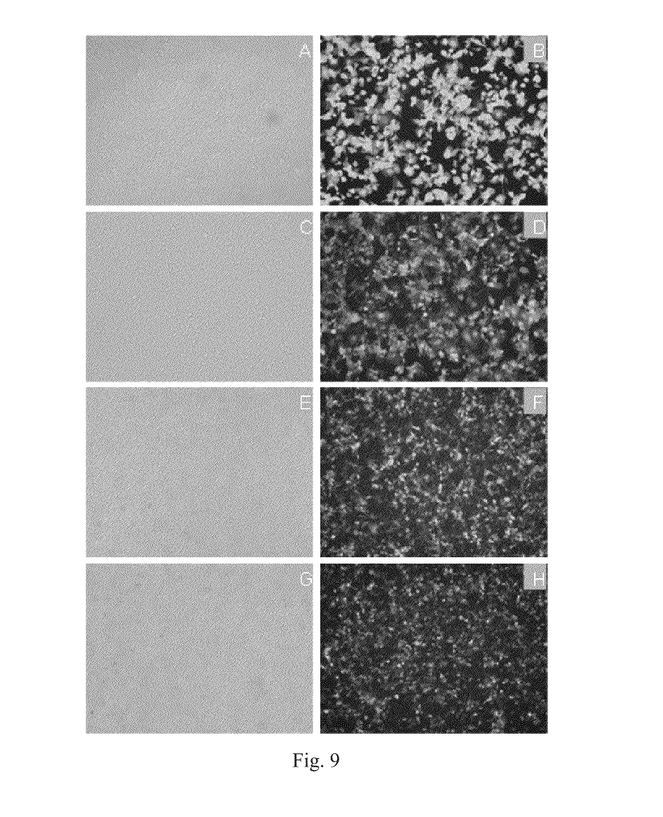

1. Hep3B, Huh7, MHCC-H and MHCC-L were separately inoculated in 35 mm culture dishes with 5×10⁵/dish, and then these cells were infected with virus MOI 100, 500, 300 and 300, respectively. After 24 h, the cultures were changed with the fresh MEM or DMEM culture containing 10% fetal calf serum, the expression of GFP were observed after 3 d. The total RNA was extracted using Trizol kit and performed reverse transcription reaction for 2 h. The diluted reverse transcription product of 1 μl was as a template to take the action of HNF1α real-time PCR amplification, in the meantime, the real-time PCR reaction with β-actin was carried out as an inner control, the reaction condition and system were same as former. The results showed that the expression of HNF1α mRNA was up-regulated obviously in human hepatocellular carcinoma cell lines infected with AdHNF1α (see FIG. 9).

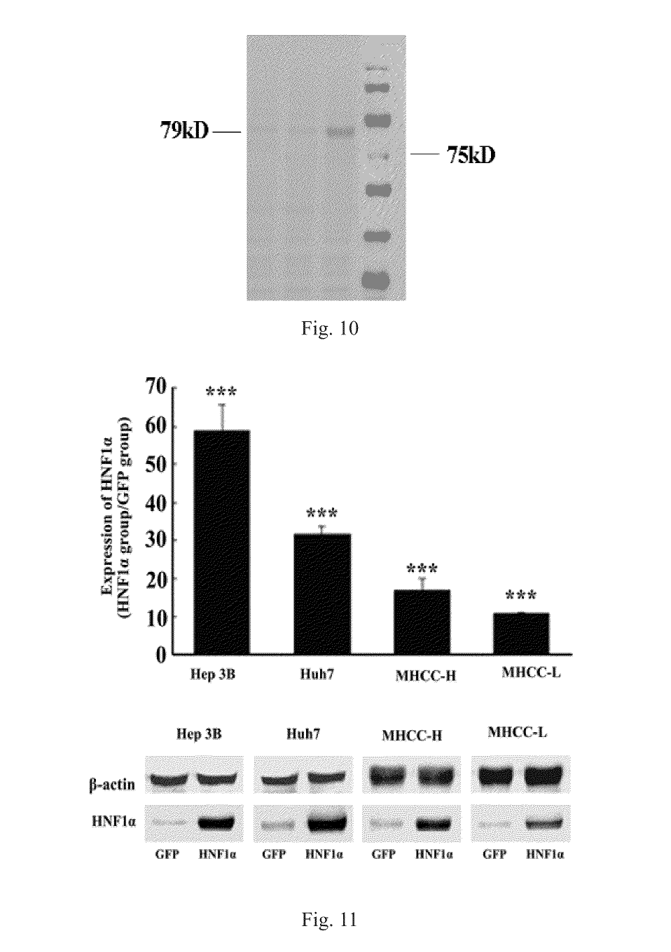

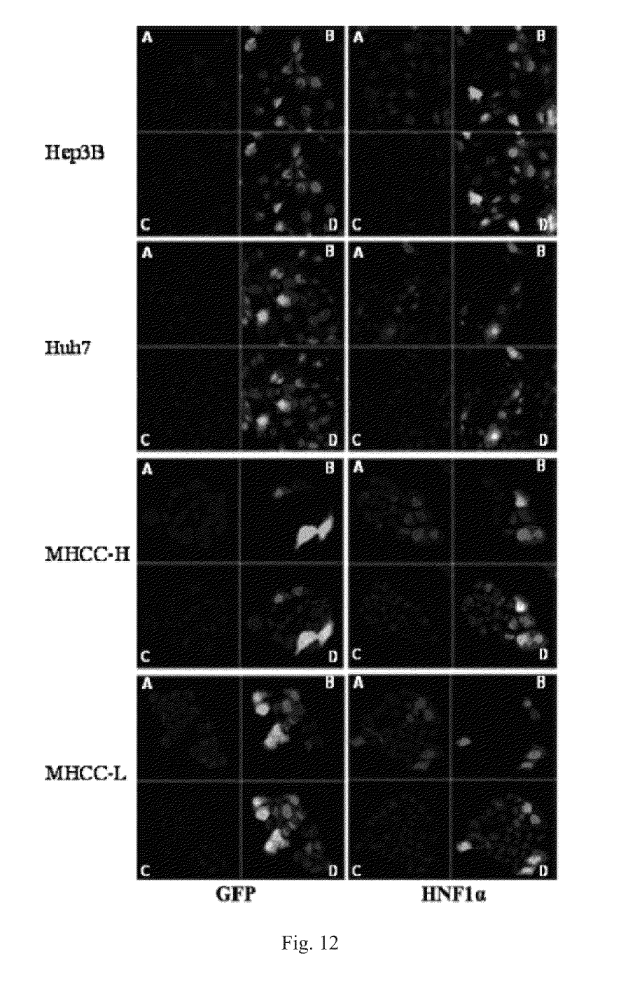

2. Hep3B, Huh7, MHCC-H and MHCC-L were infected with AdHNF1α, respectively. The whole cell protein was collected from cell lysate, after standard quantification, the protein of 10 μg was separated by 10% SDS-PAGE electrophoresis. The PVDF membrane was washed with ddH₂O. After the running gel, PVDF membrane and filter paper were balanced in Transferring Buffer, these materials were placed in electrontransfer groove under 18V for 40 min. The membrane was closed using 5% BSA/PBST of 20 ml at room temperature for 2 h. The HNF1α monoclonal antibody (1:200) was incubated at 4° C. for overnight and washed by PBST next day, and then incubated with donkey resistance to sheep fluorescence secondary antibodies (1:2000) at room temperature for 30 min. After washed two times by PBST, the fluorescence and gray scanning were performed by Odyssey infrared laser imaging system. The results showed that the expression of HNF1α protein was up-regulated obviously in human hepatocellular carcinoma cell lines infected with AdHNF1α (see FIG. 10).

3. Cyto-immunofluorescence: the sheet glasses were soaked in 75% ethanol and burned by alcohol lamp, after cooling, they were placed in 35 mm culture dishes. Hep3B, Huh7, MHCC-H and MHCC-L were inoculated in 35 mm culture dishes with 5×10⁵/dish, and then these cells were infected with virus MOI 100, 500, 300 and 300, respectively. The expression of GFP was observed after 3 d. The cells were washed two times by pre-cooled PBS and added with 4% PFA (w/v)/0.1% Triton-X-100/PBS of 1 ml for mounting at 4° C. for 30 min. The specimen was washed three times by 0.05% PBST and closed in 5% horse serum wet box at room temperature for 2 h. The confining liquid was removed from the culture dish. A box was sketched along around the cover glass using a crayon and a line was painted along the middle line of the cover glass to obtain two uneven areas. 5% horse serum was added into one area and diluted monoclonal antibody solution (1:200, HNF1α monoclonal antibody solution was prepared with confining liquid) was added into another area. The system was incubated in wet box at 4° C. for overnight. In the next day, the specimen was washed three times by 0.05% PBST, the diluted secondary antibodies solution (1:500, donkey resistance to rabbit marked with cy2 was prepared with confining liquid) was added into and the system was incubated in wet box at room temperature for 30 min. After washed by PBST, 30 μl mounting solution containing color reagent of nuclear DNA was dropped onto the specimen. The expression and localization of HNF1α were observed using a confocal microscopy after mounted by enamel and dry in air. The results displayed that the expression of HNF1α was improved obviously after infected with AdHNF1α, and located mainly in nucleus as shown in FIG. 11 and FIG. 12.

### Example 6

### The Influence in Related Functional Gene of Human Hepatoma Cell and Hepatic Cell Introduced with Exogenous HNF1α

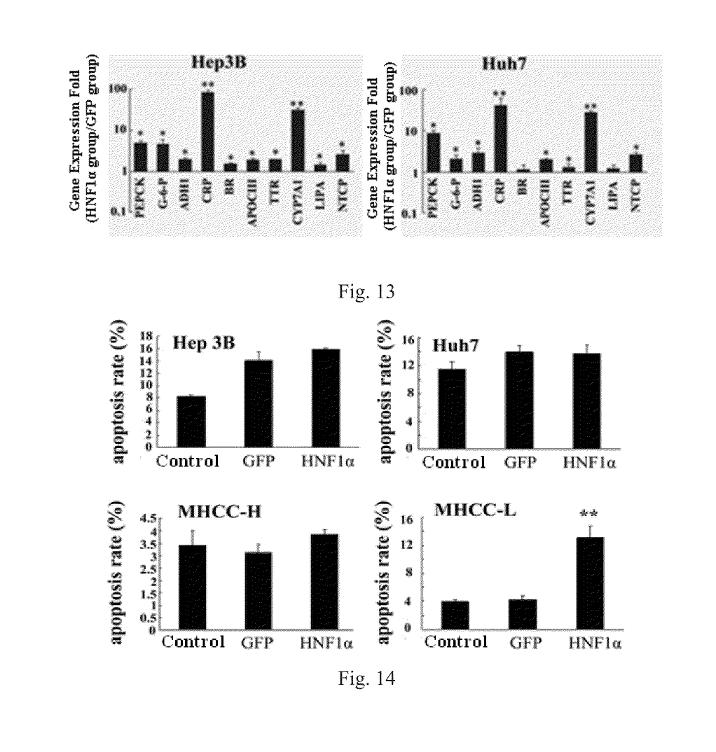

The expression of related functional gene of hepatic cell was detected by Real-time RT-PCR: Hep3B, Huh7, MHCC-H and MHCC-L were inoculated in 35 mm culture dishes with 5×10⁵/dish, and then these cells were infected with virus MOI 100, 500, 300 and 300, respectively. After 24 h, the culture were changed with the fresh MEM or DMEM culture containing 10% fetal calf serum, the expression of GFP were observed after 3 d. The total RNA was extracted using Trizol kit and performed reverse transcription reaction for 2 h. The diluted reverse transcription product of 1 μl was as a template to take the action of HNF1α real-time PCR amplification, the reaction condition and system were same as former, the primer sequence was showed in Table 4. The results showed that the expression of related functional gene in parts of hepatic cells infected with AdHNF1α was up-regulated obviously compared with that of control group, mainly including the glucose-6-phosphatase (G-6-P), the alcohol dehydrogenase 1 (ADH1), the biliverdin reductase (BR), the apolipoprotein CIII (APOCIII), the transthyretin (TTR), the phosphoenolpyruvate carboxykinase (PEPCK), the C-reactive protein (CRP), the cytochrome P450 7A1 (CYP7A1), the Na⁺/taurocholate co-transporter (NTCP), the lipase A (LIPA) and so on. In Hep3B and Huh7, G-6-P were up-regulated 4.67±1.18 fold (P<0.05) and 2.03±0.51 fold (P<0.05), respectively; ADH1 were up-regulated 1.91±0.24 fold (P<0.05) and 2.91±0.94 fold (P<0.05), respectively; BR were up-regulated 1.52±0.13 fold (P<0.05) and 1.12±0.33 fold, respectively; APOCIII were up-regulated 1.90±0.18 fold (P<0.05) and 1.97±0.17 fold (P<0.05), respectively; TTR were up-regulated 1.91±0.05 fold (P<0.05) and 1.32±0.25 fold (P<0.05), respectively; PEPCK were up-regulated 4.90±0.65 fold (P<0.01) and 8.91±1.36 fold (P<0.05), respectively; CRP were up-regulated 83.65±13.06 fold (P<0.01) and 42.68±18.07 fold (P<0.01), respectively; CYP7A1 were up-regulated 31.23±3.33 fold (P<0.01) and 27.44±3.15 fold (P<0.01), respectively; LIPA were up-regulated 1.42±0.22 fold (P<0.05) and 1.22±0.24 fold (P<0.05), respectively; NTCP were up-regulated 2.60±0.56 fold (P<0.05) and 2.65±0.32 fold (P<0.05). Other related functional gene in hepatic cells, for example, ALB, GS, CYP1A2, CYP2E, APOA2 and INSR were up-regulated non-obviously (P>0.05), as shown in FIG. 13.

TABLE 4
The primer sequence of related functional gene in
hepatic cells

| Gene | Primer sequence |
|---|---|
| AP0CIII | Sense strand 5′-GGGTACTCCTTGTTGTTGC-3′ (SEQ ID NO. 7) |
| | Antisense strand 5′-AAATCCCAGAACTCAGAGAAC-3′ (SEQ ID NO. 8) |
| G-6-P | Sense strand 5′-GGCTCCATGACTGTGGGATC-3′ (SEQ ID NO. 9) |
| | Antisense strand 5′-TTCAGCTGCACAGCCCAGAA-3′ (SEQ ID NO. 10) |
| ALB | Sense strand 5′-AGCCTAAGGCAGCTTGACTT-3′ (SEQ ID NO. 11) |
| | Antisense strand 5′-CTCGATGAACTTCGGGATGA-3′ (SEQ ID NO. 12) |
| GS | Sense strand 5′-CCTGCTTGTATGCTGGAGTC-3′ (SEQ ID NO. 13) |
| | Antisense strand 5′-GAAAAGTCGTTGATGTTGGA-3′ (SEQ ID NO. 14) |
| CYP1A2 | Sense strand 5′-CTGGCCTCTGCCATCTTCTG-3′ (SEQ ID NO. 15) |
| | Antisense strand 5′-TTAGCCTCCTTGCTCACATGC-3′ (SEQ ID NO. 16) |
| PEPCK | Sense strand 5′-GTGTCCCTCTAGTCTATGAAGC-3′ (SEQ ID NO. 17) |
| | Antisense strand 5′-ATTGACTTGATCCTCCAGATAC-3′ (SEQ ID NO. 18) |
| TTR | Sense strand 5′-GCGGGACTGGTATTTGTGTCTG-3′ (SEQ ID NO. 19) |
| | Antisense strand 5′-TTAGTGACGACAGCCGTGGTG-3′ (SEQ ID NO. 20) |
| AFP | Sense strand 5′-AGCTTGGTGGTGGATGAAAC-3′ (SEQ ID NO. 21) |
| | Antisense strand 5′-CCCTCTTCAGCAAAGCAGAC-3′ (SEQ ID NO. 22) |
| CYP2E | Sense strand 5′-CGTCATAGCCGACATCCT-3′ (SEQ ID NO. 23) |
| | Antisense strand 5′-CTCCATTTCCACGAGCAG-3′ (SEQ ID NO. 24) |
| AP0A2 | Sense strand 5′-AGAAGGTCAAGAGCCCAGAG-3′ (SEQ ID NO. 25) |
| | Antisense strand 5′-TCCAAGTTCCACGAAATAGC-3′ (SEQ ID NO. 26) |
| INSR | Sense strand 5′-GCTTGCGACACTTCACG-3′ (SEQ ID NO. 27) |
| | Antisense strand 5′-TCACTTCATACAGCACGATC-3′ (SEQ ID NO. 28) |
| CYP7A2 | Sense strand 5′-GCTTGCGACACTTCACG-3′ (SEQ ID NO. 29) |
| | Antisense strand 5′-TCACTTCATACAGCACGATC-3′ (SEQ ID NO. 30) |
| LIPA | Sense strand 5′-GCTTGCGACACTTCACG-3′ (SEQ ID NO. 31) |
| | Antisense strand 5′-TCACTTCATACAGCACGATC-3′ (SEQ ID NO. 32) |

### Example 7

### The Influence in Apoptosis and Cell Cycle of Human Hepatoma Cells Introduced with Exogenous HNF1α

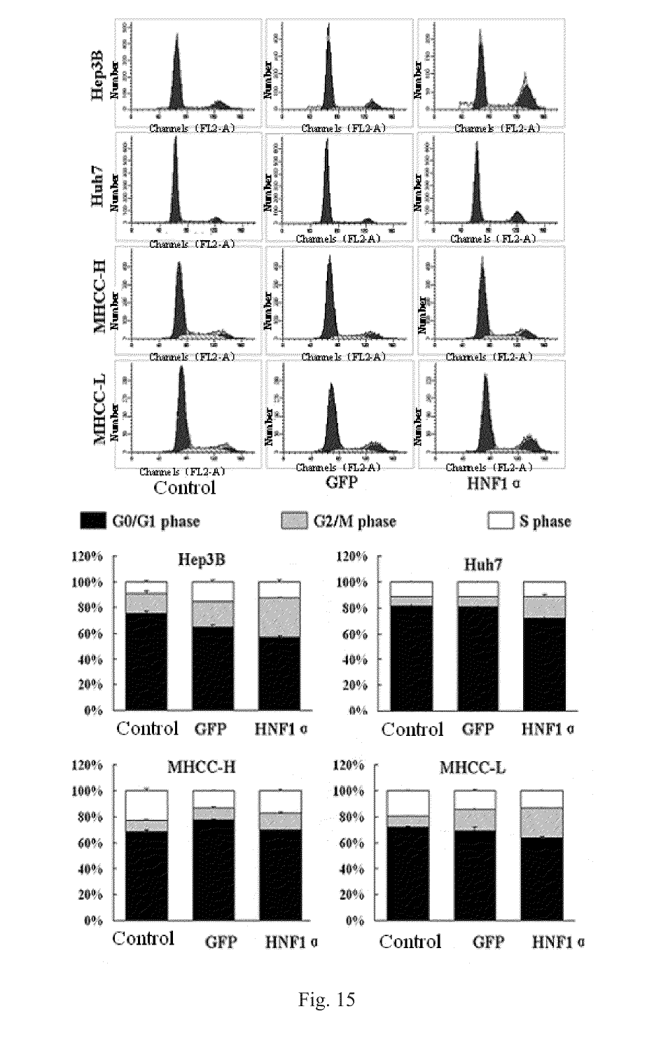

1. The apoptosis rate of human hepatoma cells was determined by a flow cytometry: Hep3B, Huh7, MHCC-H and MHCC-L were inoculated in 35 mm culture dishes with 5×10⁵/dish, and then these cells were infected with virus MOI 100, 500, 300 and 300, respectively. After 24 h, the cultures were changed with the fresh MEM or DMEM culture containing 10% fetal calf serum. The cells were collected in the third day; the apoptosis rate was determined by an EPICS XL flow cytometry and statistical analysis was performed subsequently. Each group was arranged two dishes and repeated three times. The results showed that: after the expression of HNF1α in hepatocellular carcinoma cells was up-regulated, the apoptosis rate of each hepatoma cell lines was increased non-obviously except MHCC-L (see FIG. 14).

2. The cell cycle variation of human hepatocellular carcinoma cells was determined by a flow cytometry: Hep3B, Huh7, MHCC-H and MHCC-L were inoculated in 35 mm culture dishes with 5×10⁵/dish, and then these cells were infected with virus MOI 100, 500, 300 and 300, respectively. After 24 h, the cultures were changed with the fresh MEM or DMEM culture containing 10% fetal calf serum. The cells were collected in the third day; the cell cycle variation was determined by an EPICS XL flow cytometry and statistical analysis was performed subsequently. Each group was arranged two dishes and repeated three times. The results showed that: after the expression of HNF1α was up-regulated, the cells of G2/M phase of each hepatoma cell lines was increased obviously compared with the carry-free virus AdGFP group and the control group, P<0.05 (see FIG. 15).

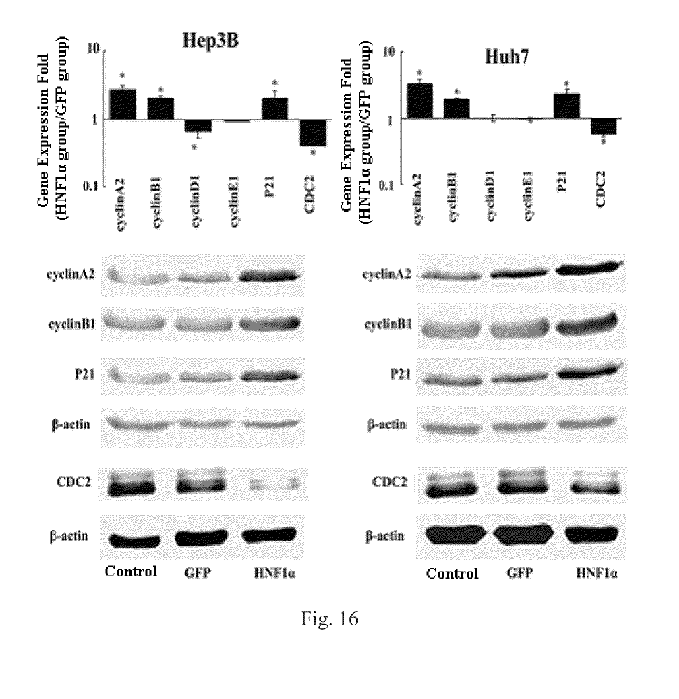

3. Real-time RT-PCR and western blotting were carried out to examine the expression of cell cycle related protein mRNA and protein: Hep3B and Huh7 were infected with AdGFP and AdHNF1α for 72 h, respectively. The total RNA was extracted using Trizol kit and the whole cell protein was collected from cell lysate. The proteins were determined by Real-time RT-PCR (the primer sequence was showed in Table 5) and western blot, respectively. The results showed that: after the expression of HNF1α was up-regulated, the cell cycle related protein cyclinA2 and cyclinB1 were up-regulated, the cell division cycle protein 2 (CDC2) was down-regulated, P21 was up-regulated (P<0.05); the changes of cyclinD and cyclinE were not statistically significant (P>0.05). The above research indicated that the inhibitory action of HNF1α to tumor may be through regulating P21 and CDC2 to leading to the G2/M phase retardation in tumor cell (see FIG. 16).

TABLE 5
The gene primer sequence of cell cycle related protein

| Gene | Primer sequence |
|---|---|
| cyclinA | Sense strand 5′-TTATTGCTGGAGCTGCCTTT-3′ (SEQ ID NO. 33) |
| 2 | Antisense strand 5′-CTCTGGTGGGTTGAGGAGAG-3′ (SEQ ID NO. 34) |
| cyclinB | Sense strand 5′-CGGGAAGTCACTGGAAACAT-3′ (SEQ ID NO. 35) |
| 1 | Antisense strand 5′-AAACATGGCAGTGACACCAA-3′ (SEQ ID NO. 36) |
| CDC2 | Sense strand 5′-AAGCCGGGATCTACCATACC-3′ (SEQ ID NO. 37) |
| | Antisense strand 5′-CCTGCATAAGCACATCCTGA-3′ (SEQ ID NO. 38) |
| P21 | Sense strand 5′-ACCGAGGCACTCAGAGGAG-3′ (SEQ ID NO. 39) |
| | Antisense strand 5′-GCCATTAGCGCATCACAGT-3′ (SEQ ID NO. 40) |
| cyclinD | Sense strand 5′-GACCTTCGTTGCCCTCTGT-3′ (SEQ ID NO. 41) |
| | Antisense strand 5′-TGAGGCGGTAGTAGGACAGG-3′ (SEQ ID NO. 42) |
| cyclinE | Sense strand 5′-GAAATGGCCAAAATCGACAG-3′ (SEQ ID NO. 43) |
| | Antisense strand 5′-GAGTTTGGGTAAACCCGGTC-3′ (SEQ ID NO. 44) |

4. The expression condition of P21 reporter gene was detected by dual-luciferase reporter gene detection system: the Hep3B cells of 2×10⁶ were inoculated into a big dish, after attached growth for 24 h, the cells density reached to 60%˜70%, the culture was changed with serum-free and antibiotic-free culture solution. P21 reporter gene expression plasmid WWP of 10 μg, internal standard plasmid SV40 of 1 μg and OPTI-MEM of 250 μl were mixed to prepare the A solution. Lipofectamine 2000 of 20 μl and OPTI-MEM of 250 μl were mixed to prepare the B solution. The A solution and the B solution were fully mixed, after placed at room temperature for 30 min, the mixed solution was added into Hep3B cells. The medium was changed with MEN culture solution containing serum after 6 h. The MEN culture solution was removed after 4 h, and the cells were washed two times by PBS and digested by 1× pancreatin for 10 min. The 1× pancreatin was neutralized by MEN solution containing serum and the cells were count and divided into 24-well plates with 1×10⁵/well. After cultivated to attached growth, the MOI 100 cells were infected with AdGFP and AdHNF1α, respectively. Each group was arranged three wells and the cells were cultivated for 72 h. The culture solution was removed and the specimen was washed by PBS, Passive Lysis Buffer of 500 μL was added into every well. The plates were shaking slightly for 15 min, and then the cell lysis solution was harvested. The cell lysis solution of 20 μl was moved to a fluorometry tube and the testing reagent luciferase from firefly of 100 μl was added into and mixed. The activity of luciferase was determined by a Fluorescence measuring Luminometer; each sample was determined three times and calculated the mean value to get the expression value of luciferase from firefly. The results showed that: after infected with virus for 72 h, the expression values of luciferase from firefly in the control group, AdGFP group and AdHNF1α group were 49.47±14.41, 59.25±17.59 and 85.3±29.21, respectively; the expression of P21 reporter gene of AdHNF1 group was up-regulated obviously in comparison with that of AdGFP group.

### Example 8

### The Influence in Proliferation of Human Solid Tumor Cells Introduced with Exogenous HNF1α

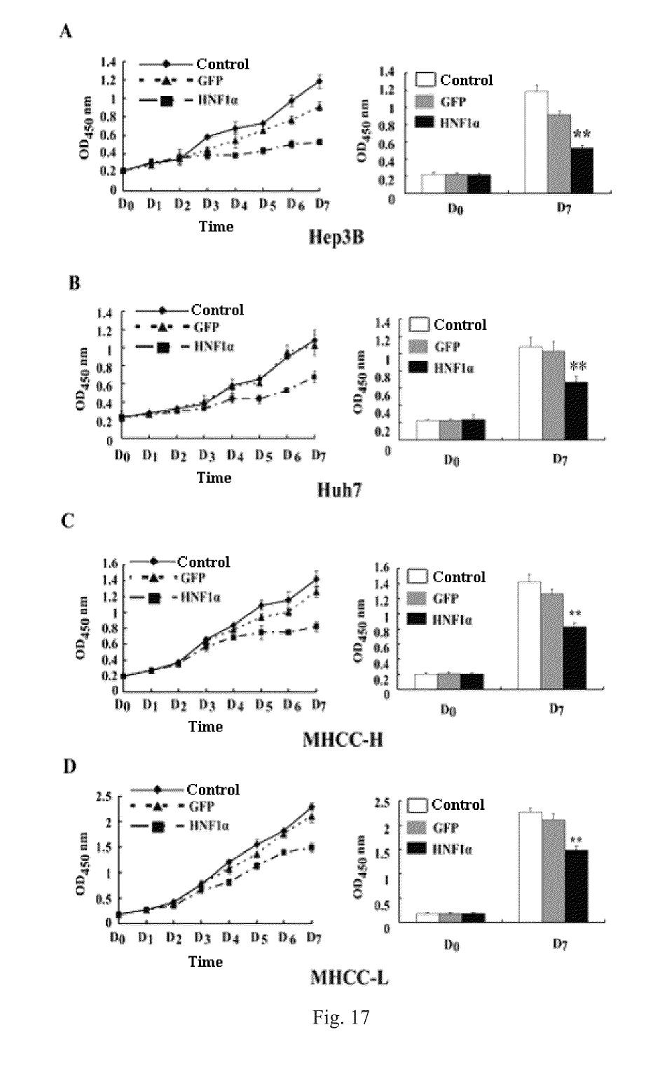

Human hepatocellular carcinoma cell lines, gastric cancer cell lines and colon cancer cell lines were inoculated in 96-well plates with 5×10³/well, respectively; and after 24 h, these cell lines were infected with virus AdHNF1α. After that, the absorbance at 450 nm wavelength was detected by CCK8 reagent every day to estimate the number of active cells. The results showed that: the expression of HNF1α has an obvious inhibitory effect on the proliferation of solid tumor cells; at the same time, the study found that, along with increasing the viral titer of virus, the inhibitory effect on the proliferation of solid tumor cells through up-regulating the expression of HNF1α manifested as time and dose dependence as shown in FIG. 17.

### Example 9

### The Influence in Colony Formation of Human Solid Tumor Cells Introduced with Exogenous HNF1α

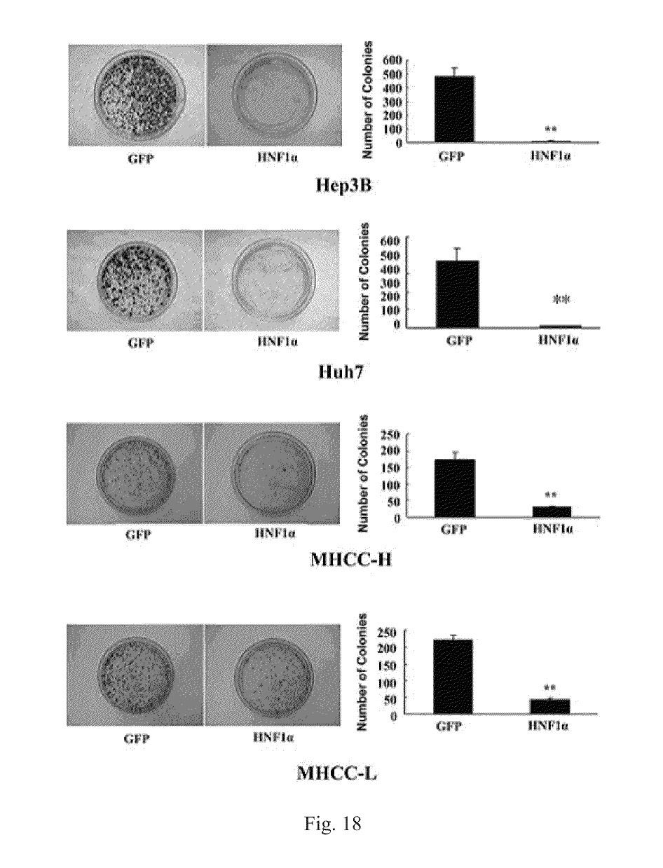

Human hepatocellular carcinoma cell lines, gastric cancer cell lines and colon cancer cell lines were inoculated in 35 mm culture dishes with 2×10⁵, respectively. After infected with AdHNF1α for 24 h, each kind of cell was inoculated in 10 mm culture dish with 8×10³. The culture solution was changed every three days and these cells were cultivated for 3-4 w until the visible cloning appeared. The cells were fixed by 4% PFA and stained by crystal violet and then the number of clone was count. The clone formed from human solid tumor cell lines was reduced in comparison with that of control group after the cells were infected with AdHNF1α, the formation ability of clone from human solid tumor cell lines was reduced through up-regulating the expression of HNF1α (see FIG. 18).

### Example 10

### The Tumorigenicity Experiment was Carried Out Through Up-Regulating the Expression of HNF1α in Human Hepatocellular Carcinoma Cell Lines Hep3B and Huh7

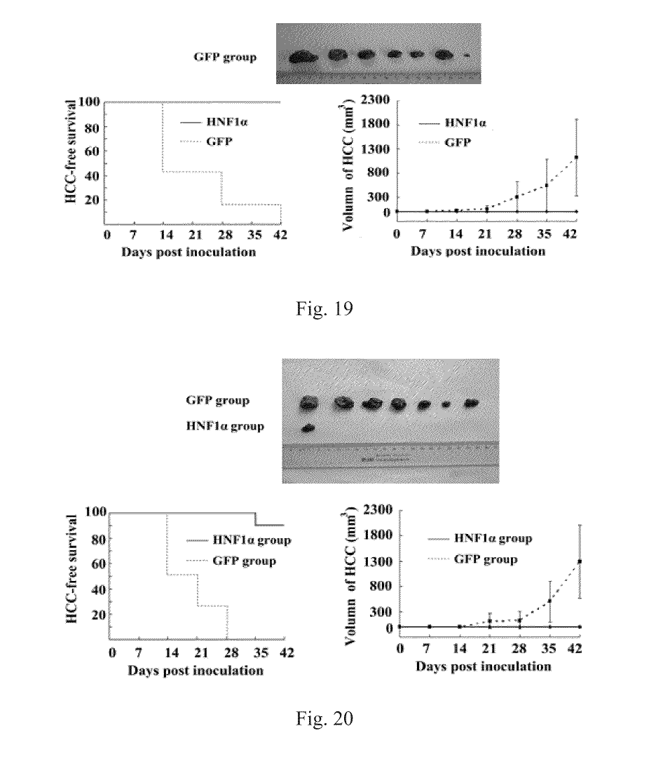

The Hep3B and Huh7 infected with AdHNF1α for 24 h were inoculated in the armpit of nude mice with 5×10⁶ and 2×10⁶, respectively. The tumors formed in vivo were observed and the size of newborn tumors was measured by vernier caliper in the meantime. The results showed that: after subcutaneous injection of Hep3B, the control side (AdGFP side) had growing tumor from the twelfth day, all of seven mice had growing tumor till the 37th day; but all of seven mice treated with AdHNF1α not had growing tumor till the 6 w (see FIG. 19). After subcutaneous injection of Huh7, the control side (AdGFP side) had growing tumor from the tenth day, all of eight mice had growing tumor till the 28th day and one mice died after 6 w; but only one mice in the treatment group had growing tumor till the 42nd day (see FIG. 20).

### Example 11

### The Experimental Hepatoma Model was Treated by HNF1α (1)

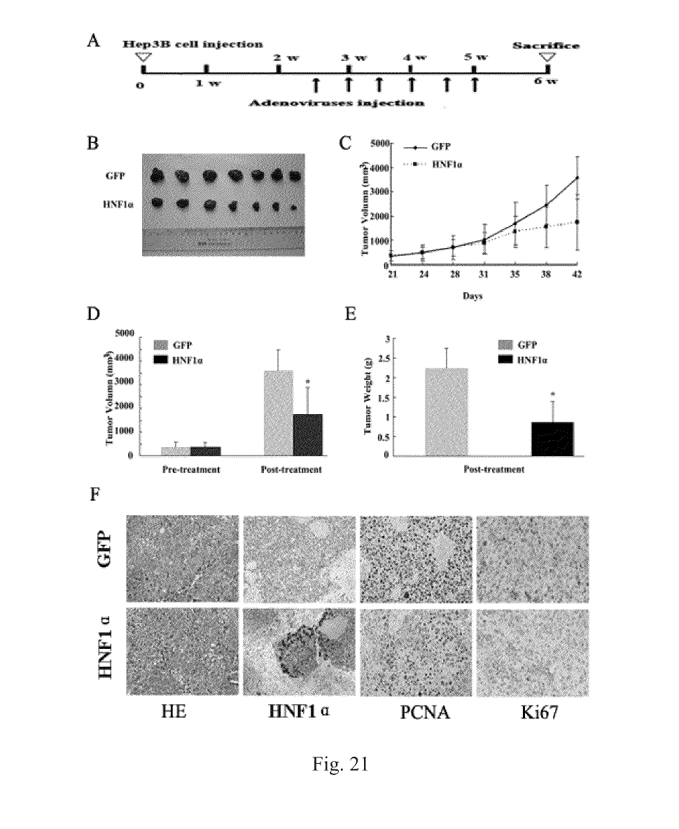

Hep3B of 5×10⁶ were resuspended in 200 μl serum-free MEM, the suspension was subcutaneously inoculated in the two sides of nude mice's neck to construct the experimental hepatoma nude mice model. Until the tumor appeared macroscopically in the two sides, the nude mouse owning tumors with the same size were selected and injected intra-tumorally with the recombinant replication-defective adenovirus carrying AdGFP and AdHNF1α in the two sides. The dose of injection was 2×10⁹ pfu and the injection was carried out three times every week for lasting 2 w. Before and after treatment and during treatment, the length diameter and width diameter of subdermal tumor was measured by a vernier caliper every week to calculate tumor volume for judging the growth condition of tumor. The nude mice were sacrificed after 6 w and the size and weight of subdermal tumor were measured. The paraffin section of tumor tissue was prepared and carried out HE staining. The expressions of HNF1α, PCNA and Ki67 of tumor tissue were detected by immunohistochemical method; the method and condition of immunohistochemistry analysis were mentioned previously. The results showed that the mean sizes of tumor in HNF1α gene-treated side were all less than that of control side at different points in time for seven mice (see FIG. 21).

### Example 12

### The Experimental Hepatoma Model was Treated by HNF1α (2)

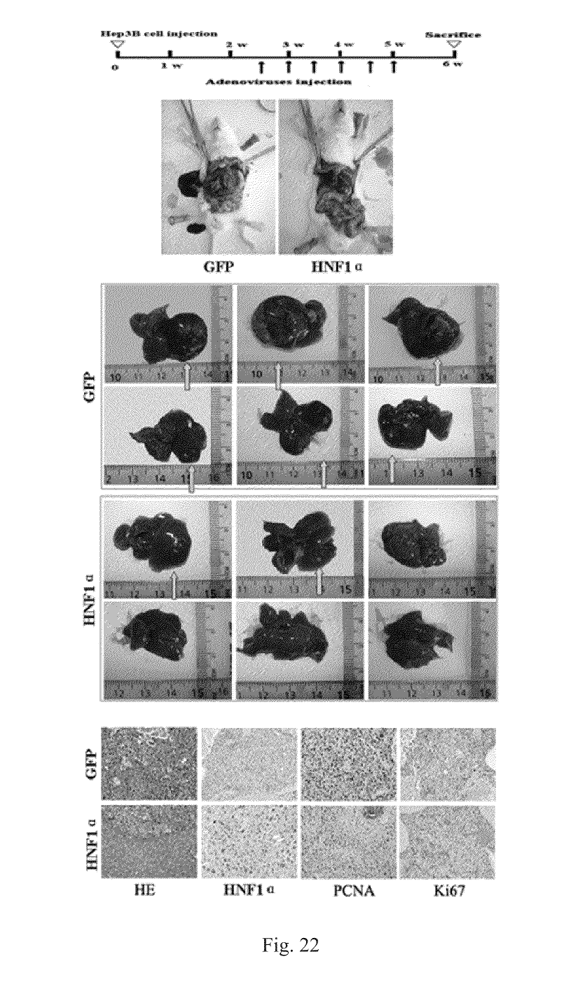

Hep3B of 5×10⁶ were resuspended in 200 μl serum-free MEM, the suspension was injected into the NOD/SCID mice through the liver in situ. After 14 days, there were white punctuate growing tumors in the injection site in liver through laparotomy operation and observation. The AdGFP and AdHNF1α were injected into coccygeal vein. The dose of injection was 2×10⁹ pfu/mouse; the injection was carried out two times every week for lasting 3 w. The mice were sacrificed, the weight of mice and liver were measured to observe the local tumor growth condition in liver. The liver was take out to prepare paraffin section and carried out HE staining and pathologic analysis. The results showed that: for AdGFP group, there were growing tumors in liver in all of six mice, in which, four mice appeared ascites and three mice appeared bloody ascites. For AdHNF1α group, there were growing tumors in liver in two mice of six mice; four mice do not appear tumor and all of six mice do not appear obvious ascites. Immunohistochemistry analysis showed that the expression of HNF1α in AdHNF1α-treated group was obviously higher than that of AdGFP-treated group, the expressions of PCNA and Ki67 were obviously lower than that of AdGFP-treated group (see FIG. 22).

1. A use of a hepatocyte nuclear factor 1α gene and/or protein in preparation of drugs for treating malignant solid tumor diseases.

2. A use of a hepatocyte nuclear factor 1α and/or protein in preparation of differentiation inducing reagents or composition for inducing differentiation of malignant solid tumor cells.

3. The use according to claim 1 or claim 2, wherein said solid tumor is selected from liver cancer, gastric cancer, intestinal cancer, pancreatic cancer, lung cancer, prostate cancer or gonad tumor.

4. The use according to claim 2, wherein said composition is pharmaceutical composition.

5. The use according to claim 4, wherein said pharmaceutical composition contains (a) the HNF1α protein, the HNF1α coding sequence or the expression vector containing said coding sequence, and (b) the acceptable carrier or excipient in pharmaceutical field.

6. The use according to claim 5, wherein said expression vector includes viral vector and non-viral vector.

7. The use according to claim 4, wherein said pharmaceutical composition is applied in preparation of drugs for restraining the formation of solid tumor in vivo.

8. The use according to claim 1 or claim 2, wherein said hepatocyte nuclear factor 1α is the human hepatocyte nuclear factor 1α.

9. A use of a recombinant expression vector containing a hepatocyte nuclear factor 1α in preparation of drugs for treating malignant solid tumor diseases.

10. A use of a recombinant expression vector containing a hepatocyte nuclear factor 1α in preparation of differentiation inducing reagents or composition for inducing differentiation of malignant solid tumor cells.

11. The use according to claim 9 or claim 10, wherein said solid tumor is selected from liver cancer, gastric cancer, intestinal cancer, pancreatic cancer, lung cancer, prostate cancer or gonad tumor.

12. The use according to claim 9 or claim 10, wherein said expression vector includes viral vector and non-viral vector.

13. A method for inducing or promoting differentiation of malignant solid tumor of mammals, wherein said method includes the following steps: the objects of mammals that need treatment are supplied with the hepatocyte nuclear factor 1α protein, the HNF1α coding sequence, or the expression vector containing said coding sequence.
